(19) United States
(12) Patent Application Publication
(10) Pub. No.: US 2024/0138753 A1
(43) Pub. Date: May 2, 2024

(54) SYSTEM AND METHOD FOR IN-VIVO INSPECTION

(71) Applicant: Given Imaging LTD, Yoqneam (IL)

(72) Inventors: Arkadiy Morgenshtein, Haifa (IL); Iddo Diukman, Zihron Yaakov (IL); Benny Linder, Kiryat Tivon (IL); Dori Peleg, Kiryat Bialik (IL)

(21) Appl. No.: 18/278,986

(22) PCT Filed: Mar. 21, 2022
(86) PCT No.: PCT/IL2022/050318

§ 371 Date: Aug. 25, 2023

Related U.S. Application Data
(60) Provisional application No. 63/163,992, filed on Mar. 22, 2021.

(51) Int. Cl. A61B 5/00; G16H 40/67; G16H 50/20

(52) U.S. Cl. CPC A61B 5/4233; A61B 5/6847; A61B 5/7221; A61B 5/7267; A61B 5/7282; G16H 40/67; G16H 50/20

(57) ABSTRACT

A system for diagnosing an esophageal disease includes at least one processor and at least one memory storing instructions. The instructions, when executed by the at least one processor, cause the system to: access, during a procedure involving an in-vivo device located within a person, data measured by the in-vivo device relating to an esophageal disease; evaluate, during the procedure while the in-vivo device is located within the person, a diagnosis for the esophageal disease for the person by applying a trained machine learning model to the data measured by the in-vivo device; and communicate, during the procedure while the in-vivo device is located within the person, the diagnosis for the esophageal disease.

### CROSS-REFERENCE TO RELATED APPLICATIONS

The present applications claims the benefit of and priority to U.S. Provisional Application No. 63/163,992, filed Mar. 22, 2021, the entire contents of which are hereby incorporated by reference herein.

### FIELD

The present disclosure generally relates to gastrointestinal (GI) tract monitoring and, more particularly, to in-vivo inspection of a patient's esophagus.

### BACKGROUND

It is well known in the art to monitor various parameters of the esophagus (e.g., pressure, pH, etc.), which are used as an indication for various pathologies. One example of such a pathology is Gastroesophageal reflux disease (GERD).

There are known devices to measure and evaluate the frequency and duration of acid reflux in order to better understand a patient's symptoms. Such devices are usually attached to the esophagus wall of the patient and are retained there over an extended period of time (e.g., up to 96 hours), while constantly monitoring the patient's pH levels. The attached device is configured to monitor, record, and transmit data to an external recorder, usually worn by the patient.

Acknowledgement of the above references herein is not to be inferred as meaning that these are in any way relevant to the patentability of the present disclosure.

### SUMMARY

Provided in accordance with aspects of the present disclosure is a system for GI inspection including an in-vivo module configured for being introduced within the GI tract of a patient for monitoring at least one parameter of the GI tract, and an ex-vivo module configured for being in proximity to the in-vivo module. The in-vivo module includes a communication unit configured for transmitting low-energy signals related to the at least one parameter to the ex-vivo module. The ex-vivo module includes a receiving unit configured for receiving the low-energy signals from the communication unit of the in-vivo module.

In an aspect of the present disclosure, the in-vivo module is configured for being affixed to the patient's GI tract and the ex-vivo module may be configured for being fitted to the patient's body at a location in proximity to the in-vivo module. Since the in-vivo module transmits low-energy signals, the quality of the reception of the signals may vary based on the proximity of the ex-vivo module to the in-vivo module.

In another aspect of the present disclosure, the in-vivo module may be configured for gathering data from the area of the GI tract at which it is located and transmit this data as low-energy signals to the ex-vivo module. It should be appreciated that since the in-vivo module relies on low-energy transmission, it is possible that some of the signals may not be properly received by the ex-vivo module, despite the minimal distance between the modules. In order to avoid this problem, the system of the present disclosure may in some aspects be configured for resending a signal to the ex-vivo module until it is properly received and only then proceed to sending a confirmation signal. This mode of operation may be specifically suited for in-vivo sensing which does not produce large amounts of data from the GI tract. More particularly, it should be understood that this is suited for arrangements in which the ratio between the amount of data produced and the habitation time of the in-vivo module within the patient is low, allowing for multiple re-sending attempts of the same signal without loss of data throughout the process.

In still another aspect of the present disclosure, the data collected by the in-vivo module may be pH readings or other non-visual data, which does not require large amounts of storage volume. In yet another aspect of the present disclosure, the in-vivo module may further include a storage component configured for storing the data before it is sent to the ex-vivo module. It should be appreciated that owing to the low amount of data produced by the in-vivo module, a memory unit with relatively low storage capacity may suffice in storing the required data.

The volume of the storage component may be designed in relation to the successful transmission rate of signals. In particular, the storage component does not have to be configured for storing all the data produced during the entire process, but rather a sufficient volume allowing lossless transmission of data. In an aspect of the present disclosure, any data which has been successfully transmitted to the ex-vivo module may be deleted from the storage component in order to free volume for additional incoming data collected by the in-vivo module.

In another aspect of the present disclosure, the in-vivo module may be configured for transmitting low-energy signals, for example, in Bluetooth Low Energy (BLE), directly to the ex-vivo module. In addition, the communication unit of the ex-vivo module may also be configured for transmitting signals to the in-vivo module. In particular, the communication unit of the ex-vivo module may be configured for confirming to the in-vivo module that a signal has been received.

In still another aspect of the present disclosure, the in-vivo module may include an anchoring arrangement configured for attaching the in-vivo module to a specific location within the GI tract. Similarly, the ex-vivo module may in some aspects of the present disclosure include a fitting mechanism configured for securely fitting the ex-vivo module to the patient.

The system may be configured for inspection of the esophageal segment of the patient's GI tract. Specifically, in an aspect of the present disclosure, the in-vivo module may be positioned in close proximity to the esophageal sphincter and in close proximity to the squamocolumnar junction or so called “Z-line”. The Z-line represents the normal esophagogastric junction where the squamous mucosa of the esophagus and columnar mucosa of the stomach meet.

In yet another aspect of the present disclosure, the ex-vivo module may be in the form of a patch configured for being adhered to the patient's skin at a given location. The patch may have an adhesive face configured for attachment to the patient and constituting the fitting mechanism, and a covering layer facing away from the patient and configured for protecting the ex-vivo module and its components. One of the advantages of an adhesive mechanism is, inter alia, its ability to affix the ex-vivo module to a specific location which can minimize displacement of the ex-vivo module with respect to the position of the in-vivo module. In aspects of the present disclosure, the ex-vivo module may include an anchoring arrangement in the form of a strap or a belt configured for being secured to the patient.

In another aspect of the present disclosure, the ex-vivo module may be a fitted to the patient at a location that is not in proximity to the in-vivo module, e.g., one of the patient's extremities. The ex-vivo module may, in some aspects, be in the form of a wearable device (e.g., a bracelet, watch, smartwatch etc.) or be fitted to the patient's body.

In still another aspect of the present disclosure, the ex-vivo module may be a hand-held device, e.g. a smartphone, which is not always in proximity to the in-vivo module. In this aspect, the ex-vivo module may be configured, when in proximity to the in-vivo module, for alerting the in-vivo module to begin transmitting data to the ex-vivo module. This arrangement allows for the power of the in-vivo module to be conserved by only transmitting data to the ex-vivo module when the ex-vivo module is in suitable proximity to the in-vivo module and can actually receive the transmitted data.

In an aspect of the present disclosure, the ex-vivo module may be configured for detecting movement of the patient based on movement of the ex-vivo module, and inferring information from the movement about patient activities related to consumption of food and digestion. For example, if the ex-vivo module is attached to the patient's hand, the ex-vivo module may be configured for inferring from the patient's hand movements that the patient is eating.

In yet another aspect of the present disclosure, movement detection of the patient may also be used for detecting sleep patterns, which may also be collated with the data obtained by the sensor of the in-vivo module.

In still yet another aspect of the present disclosure, the system may include a processor configured for collating the data obtained about the patient's movements with the data obtained from the in-vivo module, thereby providing a better understanding of the patient's GI operation. This may also eliminate the need for the patient to manually input their feeding times.

In another aspect of the present disclosure, there is provided an in-vivo module including an anchoring mechanism configured for retaining the in-vivo module at a given location within the GI tract, a sensing arrangement configured for collecting data from the GI, a low-energy communication unit configured for sending the data to an ex-vivo module in the form of low-energy signals, and a storage unit configured for storing at least part of the data.

In yet another aspect of the present disclosure, there is provided an ex-vivo module including an adhesive surface configured for attachment to a patient's skin at a given location and a communication unit configured for receiving low-energy signals from an in-vivo module.

Provided in accordance with aspects of the present disclosure is a system for a patient's GI inspection. The system includes an in-vivo module configured for being introduced within the GI tract of a patient for monitoring at least one parameter of the GI tract. The in-vivo module includes a first communication unit configured at least for sending out signals relating to the at least one parameter. The system also includes an intermediate module configured for being in proximity to the in-vivo module. The intermediate module incudes a second communication unit configured for receiving the signals from the in-vivo module and sending out the signals. The system also includes an ex-vivo module associated with the patient. The ex-vivo module includes a third communication unit configured at least for receiving the signals from the intermediate module. At least one of the communication between the in-vivo module and the intermediate module or the communication between the intermediate module and the ex-vivo module is performed via low energy transmission.

Provided in accordance with aspects of the present disclosure is a system for GI inspection. The system includes an in-vivo module configured for being introduced within the GI tract of a patient for monitoring at least one parameter of the GI tract. The system also includes a movement detection module configured for being fitted to the patient for monitoring movement thereof and an ex-vivo module configured for communicating at least with the in-vivo module. The system also includes a processor configured for collating the data received from the in-vivo module and the data received from the movement detection module.

In an aspect of the present disclosure, the movement detection module may be in the form of a wearable device fitted to the patient. The wearable device may be configured for detecting movement of the patient as a whole, and/or being fitted to a limb of a patient (arm/leg) and detecting movement of the limb.

In another aspect of the present disclosure, the ex-vivo module may be any one of: a patch, a wearable device, a fitted device, or a smartphone.

In yet another aspect of the present disclosure, the movement detection module may be any one of: a wearable device or a smartphone.

According to aspects of the present disclosure, various combinations and configurations of communication between the modules may be implemented, examples of which include, but are not limited to: direct communication between the in-vivo module and the smartphone; direct communication between the smartphone and the in-vivo module for receiving GI data; direct communication between the smartphone and the wearable device for receiving movement data; direct communication between the in-vivo module and the wearable device, wherein the wearable device collates the GI data with the movement data; direct communication between the in-vivo module and the patch; and/or direct communication between the patch and the wearable device and/or smartphone.

In accordance with aspects of the present disclosure, a system for diagnosing an esophageal disease includes at least one processor and at least one memory storing instructions. The instructions, when executed by the at least one processor, cause the system to: access, during a procedure involving an in-vivo device located within a person, data measured by the in-vivo device relating to an esophageal disease; evaluate, during the procedure while the in-vivo device is located within the person, a diagnosis for the esophageal disease for the person by applying a trained machine learning model to the data measured by the in-vivo device; and communicate, during the procedure while the in-vivo device is located within the person, the diagnosis for the esophageal disease.

In various embodiments of the system, the instructions, when executed by the at least one processor, further cause the system to: access, during the procedure while the in-vivo device is located within a person, event information relating to events of the person which occur during the procedure. Evaluating the diagnosis for the esophageal disease for the person includes applying the trained machine learning model to the data measured by the in-vivo device and to the event information.

In various embodiments of the system, accessing the event information relating to events of the person which occur during the procedure includes receiving the event information from a mobile device of the person, where at least a portion of the event information is not entered by the person and is generated by at least one of: the mobile device of the person or a wearable device separate from the mobile device.

In various embodiments of the system, the trained machine learning model includes a trained deep learning neural network configured to be applied to data collected over a predetermined time duration that is less than twenty-four hours. The data measured by the in-vivo device includes data measured by the in-vivo device over at least the predetermined time duration, such that the trained deep learning neural network is applied to the data measured by the in-vivo device over at least the predetermined time duration.

In various embodiments of the system, the trained machine learning model is one model among a plurality of trained machine learning models. The models of the plurality of trained machine learning models are configured to be applied to data collected by the in-vivo device over different predetermined time durations.

In various embodiments of the system, in evaluating the diagnosis for the esophageal disease for the person, the instructions, when executed by the at least one processor, cause the system to: evaluate, at a first time during the procedure while the in-vivo device is located within the person, a first diagnosis for the esophageal disease for the person using a first model of the plurality of trained machine learning models; determine that the first diagnosis does not meet confidence criteria; evaluate, at a second time during the procedure while the in-vivo device is located within the person, a second diagnosis for the esophageal disease for the person using the trained machine learning model, where the second time is after the first time; determine that the second diagnosis meets confidence criteria; and provide the second diagnosis as the diagnosis for the esophageal disease for the person.

In accordance with aspects of the present disclosure, a computer-implemented method for diagnosing an esophageal disease includes: accessing, during a procedure involving an in-vivo device located within a person, data measured by the in-vivo device relating to an esophageal disease; evaluating, during the procedure while the in-vivo device is located within the person, a diagnosis for the esophageal disease for the person by applying a trained machine learning model to the data measured by the in-vivo device; and communicating, during the procedure while the in-vivo device is located within the person, the diagnosis for the esophageal disease.

In various embodiments of the computer-implemented method, the method further includes: accessing, during the procedure while the in-vivo device is located within a person, event information relating to events of the person which occur during the procedure. Evaluating the diagnosis for the esophageal disease for the person includes applying the trained machine learning model to the data measured by the in-vivo device and to the event information.

In various embodiments of the computer-implemented method, accessing the event information relating to events of the person which occur during the procedure includes receiving the event information from a mobile device of the person, where at least a portion of the event information is not entered by the person and is generated by at least one of: the mobile device of the person or a wearable device separate from the mobile device.

In various embodiments of the computer-implemented method, the trained machine learning model includes a trained deep learning neural network configured to be applied to data collected over a predetermined time duration that is less than twenty-four hours. The data measured by the in-vivo device includes data measured by the in-vivo device over at least the predetermined time duration, such that the trained deep learning neural network is applied to the data measured by the in-vivo device over at least the predetermined time duration.

In various embodiments of the computer-implemented method, the trained machine learning model is one model among a plurality of trained machine learning models. The models of the plurality of trained machine learning models are configured to be applied to data collected by the in-vivo device over different predetermined time durations.

In various embodiments of the computer-implemented method, evaluating the diagnosis for the esophageal disease for the person includes: evaluating, at a first time during the procedure while the in-vivo device is located within the person, a first diagnosis for the esophageal disease for the person using a first model of the plurality of trained machine learning models; determining that the first diagnosis does not meet confidence criteria; evaluating, at a second time during the procedure while the in-vivo device is located within the person, a second diagnosis for the esophageal disease for the person using the trained machine learning model, where the second time is after the first time; determining that the second diagnosis meets confidence criteria; and providing the second diagnosis as the diagnosis for the esophageal disease for the person.

In accordance with aspects of the present disclosure, a computer-readable medium includes instructions which, when executed by at least one processor of a system, cause the system to: access, during a procedure involving an in-vivo device located within a person, data measured by the in-vivo device relating to an esophageal disease; evaluate, during the procedure while the in-vivo device is located within the person, a diagnosis for the esophageal disease for the person by applying a trained machine learning model to the data measured by the in-vivo device; and communicate, during the procedure while the in-vivo device is located within the person, the diagnosis for the esophageal disease.

In various embodiments of the computer-readable medium, the instructions, when executed by the at least one processor, further cause the system to: access, during the procedure while the in-vivo device is located within a person, event information relating to events of the person which occur during the procedure. Evaluating the diagnosis for the esophageal disease for the person includes applying the trained machine learning model to the data measured by the in-vivo device and to the event information.

In various embodiments of the computer-readable medium, the trained machine learning model includes a trained deep learning neural network configured to be applied to data collected over a predetermined time duration that is less than twenty-four hours. The data measured by the in-vivo device includes data measured by the in-vivo device over at least the predetermined time duration, such that the trained deep learning neural network is applied to the data measured by the in-vivo device over at least the predetermined time duration.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a schematic view of a GI tract monitoring system in accordance with the present disclosure, shown fitted to a patient;

FIG. 1B is a schematic enlarged view of the system shown in FIG. 1A;

FIG. 2 is a schematic block diagram of the operation process of the system shown in FIGS. 1A and 1B;

FIG. 3 is a schematic view of another GI tract monitoring system in accordance with the present disclosure;

FIG. 4 is a schematic view of another GI tract monitoring system in accordance with the present disclosure;

FIG. 5 is a block diagram of exemplary components of a device or system, in accordance with aspects of the present disclosure;

FIG. 6 is a diagram of exemplary devices and systems and communications between the devices and systems, in accordance with aspects of the present disclosure;

FIG. 7 is a diagram of an exemplary communication path between an ex-vivo device and a cloud system via a mobile hotspot, in accordance with aspects of the disclosure;

FIG. 8 is a diagram of exemplary communication paths between an ex-vivo device and a cloud system, in accordance with aspects of the disclosure;

FIG. 9 is a diagram of an exemplary communication path between an ex-vivo device and a cloud system via a healthcare provider workstation and router, in accordance with aspects of the disclosure;

FIG. 10 is a diagram of exemplary connections between an ex-vivo device and various devices, in accordance with aspects of the disclosure;

FIG. 11 is a diagram of exemplary communication paths between an ex-vivo device and healthcare provider devices, in accordance with aspects of the disclosure;

FIG. 12 is a diagram of an exemplary machine learning model, in accordance with aspects of the present disclosure; and

FIG. 13 is a flow diagram of an exemplary operation, in accordance with aspects of the present disclosure.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION

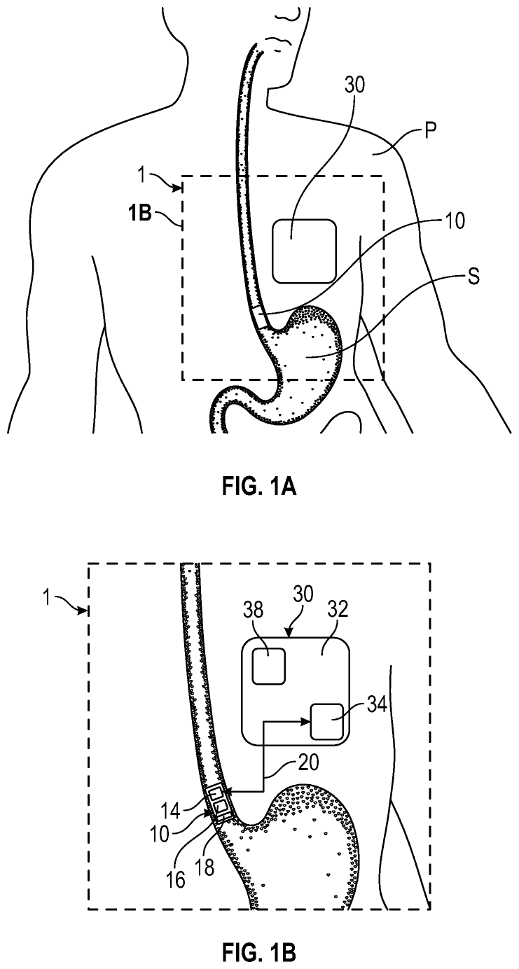

Attention is first drawn to FIGS. 1A and 1B, in which a system is shown, generally designated 1, configured for monitoring at least one parameter of a patient's GI tract. The system 1 includes an in-vivo module 10 and an ex-vivo module 30.

As used herein, and unless indicated otherwise, the term “module” may be interchangeable with the terms “device” or “system” or a similar term, but may be limited thereto. Additionally, depending on the context, the term “unit” may be interchangeable with one or more of the following terms: device, hardware, and/or circuitry, or a similar term, but may not be limited thereto. It is intended that any disclosure herein using one of the above-mentioned terms shall also be treated as a disclosure using any of the interchangeable terms for the term that is used. All such disclosure is intended and contemplated to be within the scope of the present disclosure.

As shown in FIG. 1A, the in-vivo module 10 is attached to the GI tract of a patient P, at a location proximal to the lower esophageal sphincter (LES), just before the entrance to the stomach S. The in-vivo module 10 is anchored to the esophageal wall as known per se, and is configured for monitoring various parameters of the GI tract related to the operation of the esophagus and the LES. The ex-vivo module 30 is fitted to the skin of the patient P at a location proximal to the location of the in-vivo module 10.

With particular attention being drawn to FIG. 1B, the in-vivo module 10 includes a body 12 accommodating therein a sensor 16 configured for sensing at least one parameter of the patient's GI tract relating to its location, a power source 18, and a first communication unit 14 configured for receiving data from the sensor 16 and transmitting the data to the ex-vivo module 30. The ex-vivo module 30, in turn, includes a body 32, a second communication unit 34, and a power source 38.

The ex-vivo module 30 is shown in FIGS. 1A and 1B in the form of an adhesive patch configured for being adhered to the patient's skin, thereby affixing the ex-vivo module to a specific location. In the present example, the location is chosen to be in close proximity to the location of the in-vivo module 10. One advantage of the ex-vivo module 30 being adhered to the skin is that its distance with respect to the in-vivo module 10, both laterally and depth-wise, is maintained throughout the procedure, thereby making communication between the modules 10, 30 more reliable.

Bi-directional communication is provided between the first communication unit 14 and the second communication unit 34, allowing the in-vivo module 10 to send data regarding the measured parameter to the ex-vivo module 30, as well as the ex-vivo module 30 to send signals back to the in-vivo module 10. The communication between the first communication unit 14 and the second communication unit 34 is performed by a low energy transmission 20, which, in the present example is a low energy Bluetooth low energy (BLE) communication. The term “procedure data” will be used to refer to data measured by the in-vivo module 10, among other data, as described below herein.

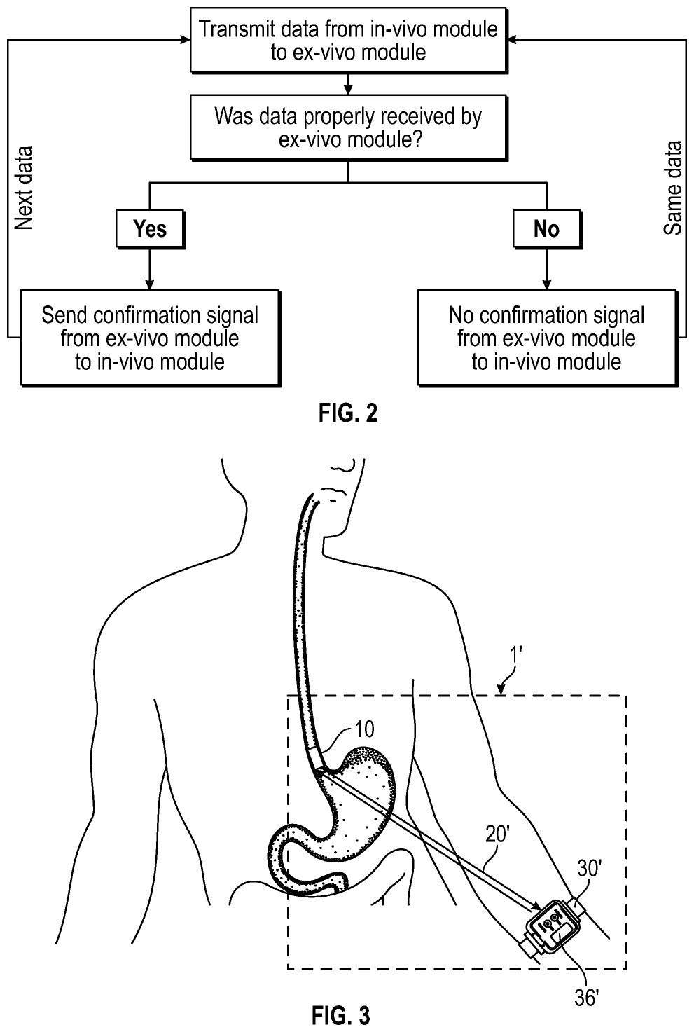

With additional attention being drawn to FIG. 2, since the communication 20 is performed by low energy transmission, it is possible that some data signals transmitted from the in-vivo module 10 may not be properly received by the ex-vivo module 30. Thus, upon establishing a transmission, the ex-vivo module 30 is configured for sending back to the in-vivo module 10 a confirmation signal indicating that data was properly received. Once such a confirmation signal is received, the in-vivo module 10 proceeds to sending the next data to the ex-vivo module 30. In the event that data is not properly received and no confirmation signal is provided to the in-vivo module 10, the in-vivo module 10 will simply attempt to transmit the same data over and over again until receiving a confirmation signal from the ex-vivo module 30.

The in-vivo module 10 may further include a storage component (not shown), configured for storing a given amount of data. The volume of the storage component is designed in proportion to the expected data which will not be properly transmitted. In other words, the in-vivo module 10 is configured for storing a sufficient amount of data based on the expected loss of data transmissions to the ex-vivo module 30.

It should be noted that for specific operations, e.g., pH monitoring, the amount of data obtained by the sensor 16 of the in-vivo module 10 does not require a large storage volume, and therefore it is even possible to store all of the data from the procedure (in the worst-case scenario where none of the data signals from the in-vivo module 10 are properly received by the ex-vivo module 30).

Attention is now drawn to FIG. 3, in which another system is shown, generally designated 1′, and including the same in-vivo module 10 as system 1, but with an ex-vivo module 30′ being in the form of a wearable device, e.g., a smartwatch or bracelet, worn on the patient's wrist. In this configuration, the low energy transmission may still be sufficient for properly transmitting the required data from the in-vivo module 10 to the ex-vivo module 30′.

The ex-vivo module 30′ may be provided with a movement sensor 36′ configured for detecting movement of the extremities, in this case the hand of the patient P. The ex-vivo module 30′ may also be provided with a processor configured for receiving data from the sensor 36′ in order to infer therefrom when the patient P is eating. Aspects of inferring an eating event based on movement sensor data are described in U.S. Pat. No. 10,790,054, which is hereby incorporated by reference herein in its entirety. As an example, an eating event may be inferred using machine learning techniques. Labeled training data (e.g., movement sensor data) may be obtained from one or more users to train a machine leaning classifier to infer whether movement sensor data indicates a food intake event is occurring or has occurred. This information can then be collated with the information obtained from the in-vivo module 10, thereby eliminating the need for the patient P to manually input their eating events. One advantage of this combination is that it addresses the problem of patients tending to manually input information post-factum, often mistaking the exact time in which they consumed food, which, in turn, makes the correlation between the eating times and the measurements received from the in-vivo module 10 more difficult.

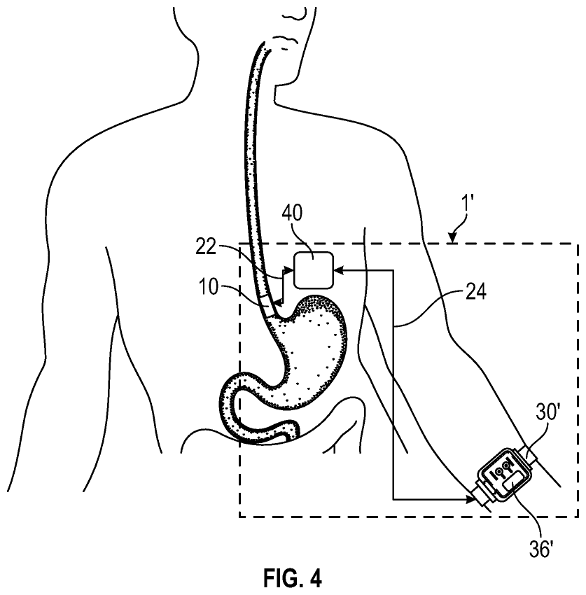

Attention is now drawn to FIG. 4, in which another system is shown, generally designated 1″, which is similar to the previously described system 1′, with the addition of an intermediate module 40, fitted to the patient. The intermediate module 40 is generally similar to the previously described ex-vivo module 30. The in-vivo module 10 is configured for bi-directional communication with the intermediate module 40, as shown by a bi-directional arrow 22, and the intermediate module 40 is configured for bi-directional communication with the ex-vivo module 30′, as shown by a bi-directional arrow 24. In accordance with different examples, at least one of the communications 22, 24, is a low energy communication as previously described. Thus, three different combinations are provided: communication 22 is a low energy transmission and communication 24 is of another type of communication (e.g., RF); communication 24 is a low energy transmission and communication 22 is of another type of communication (e.g., RF); and both communication 22, 24 are low energy transmissions.

Accordingly, described above are systems and methods relating to monitoring at least one parameter of a GI tract. The following will describe further systems and devices and communications between systems and devices.

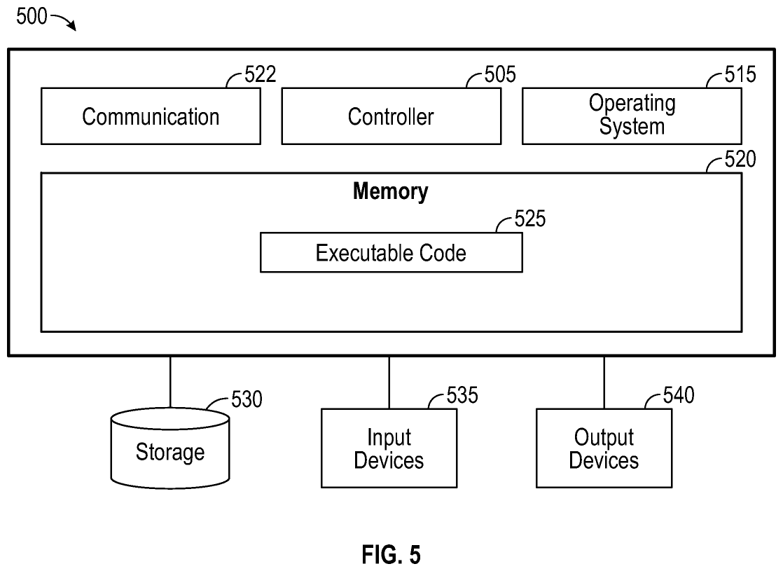

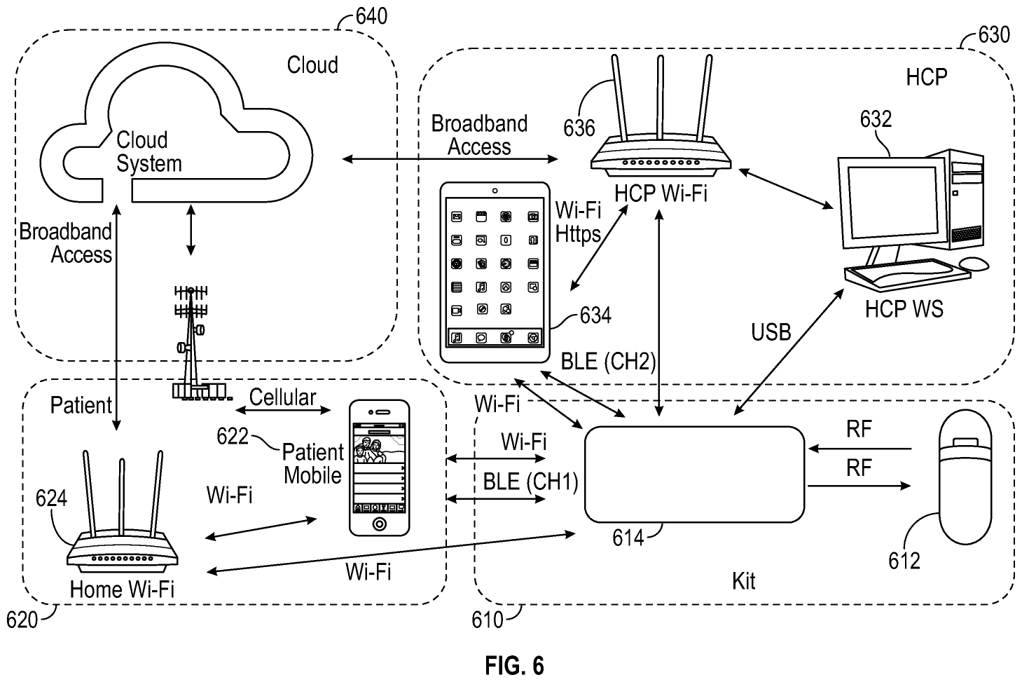

FIG. 5 shows a block diagram of exemplary components of a system or device 500. The block diagram is provided to illustrate possible implementations of various parts of the disclosed systems and devices. For example, the components of FIG. 5 may implement a patient mobile device (e.g., 622, FIG. 6) or may implement a portion of a remote computing system (e.g., 640, FIG. 6), or may implement a healthcare provider device (e.g., 632, 634, FIG. 6).

The computing system 500 includes a processor or controller 505 that may be or include, for example, one or more central processing unit processor(s) (CPU), one or more Graphics Processing Unit(s) (GPU or GPGPU), and/or other types of processor, such as a microprocessor, digital signal processor, microcontroller, programmable logic device (PLD), field programmable gate array (FPGA), or any suitable computing or computational device. The computing system 500 also includes an operating system 515, a memory 520, a storage 530, input devices 535, output devices 540, and a communication device 522. The communication device 522 may include one or more transceivers which allow communications with remote or external devices and may implement communications standards and protocols, such as cellular communications (e.g., 3G, 4G, 5G, CDMA, GSM), Ethernet, Wi-Fi, Bluetooth, low energy Bluetooth, Zigbee, Internet-of-Things protocols (such as mosquito MQTT), and/or USB, among others.

The operating system 515 may be or may include any code designed and/or configured to perform tasks involving coordination, scheduling, arbitration, supervising, controlling or otherwise managing operation of computing system 500, such as scheduling execution of programs. The memory 520 may be or may include, for example, one or more Random Access Memory (RAM), read-only memory (ROM), flash memory, volatile memory, non-volatile memory, cache memory, and/or other memory devices. The memory 520 may store, for example, executable instructions that carry out an operation (e.g., executable code 525) and/or data. Executable code 525 may be any executable code, e.g., an app/application, a program, a process, task or script. Executable code 525 may be executed by controller 505.

The storage 530 may be or may include, for example, one or more of a hard disk drive, a solid state drive, an optical disc drive (such as DVD or Blu-Ray), a USB drive or other removable storage device, and/or other types of storage devices. Data such as instructions, code, procedure data, and medical images, among other things, may be stored in storage 530 and may be loaded from storage 530 into memory 520 where it may be processed by controller 505. The input devices 535 may include, for example, a mouse, a keyboard, a touch screen or pad, or another type of input device. The output devices 540 may include one or more monitors, screens, displays, speakers and/or other types of output devices.

The illustrated components of FIG. 5 are exemplary and variations are contemplated to be within the scope of the present disclosure. For example, the numbers of components may be greater or fewer than as described and the types of components may be different than as described. When the system 500 implements a machine learning system, for example, a large number of graphics processing units may be utilized. When the computing system 500 implements a data storage system, a large number of storages may be utilized. As another example, when the computing system 500 implements a server system, a large number of central processing units or cores may be utilized. Other variations and applications are contemplated to be within the scope of the present disclosure.

Referring to FIG. 6, there is a diagram of various devices and systems of a computing configuration and communications between the devices and systems. The systems include a kit 610 that includes an in-vivo device 612 and an ex-vivo device 614, a patient system 620 that includes an Internet-enabled mobile device 622 and/or a wireless router 624, a healthcare provider system 630 that includes a computer/workstation 632, a tablet device 634, and/or a wireless router 636, and a remote computing system 640. For convenience, the remote computing system 640 is illustrated as a cloud system and may be referred to as a cloud system. However, it will be understood that the description below relating to the cloud system shall apply to other variations of a remote computing system.

In the kit 610, the in-vivo device 612 and the ex-vivo device 614 can communicate with each other using radio frequency (RF) transceivers. Persons skilled in the art will understand how to implement RF transceivers and associated electronics for interfacing with RF transceivers. In various embodiments, the RF transceivers can be designed to use frequencies that experience less interference or no interference from common communications devices, such as cordless phones, for example.

The ex-vivo device 614 can include various communication capabilities, including low energy Bluetooth (BLE), Wi-Fi, and/or a USB connection. The term Wi-Fi includes Wireless LAN (WLAN), which is specified by IEEE 802.11 family of standards. The Wi-Fi connection allows the ex-vivo device 614 to upload procedure data to the cloud system 640. The ex-vivo device 614 can connect to a Wi-Fi network in either a patient's network system 620 or a healthcare provider's network system 630, and the procedure data is then transferred to the cloud system 640 through the Internet infrastructure. The ex-vivo device 614 may be equipped with a wired USB channel for transferring procedure data when a Wi-Fi connection is not available or when procedure data could not all be communicated using Wi-Fi. The Bluetooth® low energy (BLE) connection may be used for control and messaging and data. Because the BLE connection uses relatively low power, BLE can be continuously-on during the entire procedure. Depending on the device and its BLE implementation, the BLE connection may support communications rates of about 250 Kbps-270 Kbps through about 1 Mbps. While some BLE implementations may support somewhat higher communication rates, a Wi-Fi connection is generally capable of providing much higher communication rates, which may be transfer rates of 10 Mbps or higher, depending on the connection quality and amount of procedure data. In various embodiments, when the amount of procedure data to be transferred is suitable for the BLE connection transfer rate, the procedure data can be transferred using the BLE connection.

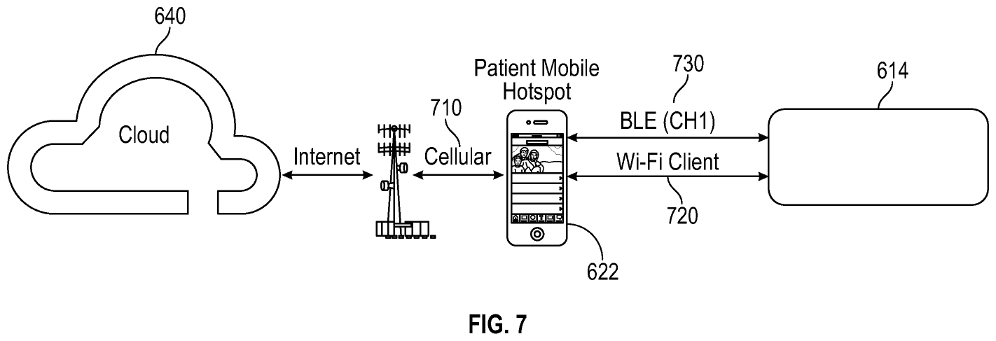

As shown in FIG. 6, there are many possible communication paths between an ex-vivo device 614 and the cloud system 640 or various devices. FIGS. 7-11 address connectivity between particular portions of FIG. 6, and they are described below. The illustrated and described embodiments are merely exemplary and other types of connections not shown or described can be used, such as Zigbee or Internet-of-Things protocols, among others.

With reference to FIG. 7, there is shown a diagram of an exemplary communication path between an ex-vivo device 614 and a cloud system 640 via tethering or mobile hotspot provided by a patient Internet-connected mobile device 622. The patient Internet-connected mobile device 622 may be referred to herein as a mobile device 622 and can include, without limitation, a smartphone, a laptop, or a tablet, among others. The mobile device 622 can be any mobile device used by a patient, including a mobile device owned by the patient or a mobile device loaned to the patient for the CE procedure. For convenience, a smartphone is illustrated in FIG. 7, but it is intended for the disclosure to apply to other types of Internet-connected mobile devices as well.

By providing tethering or a mobile hotspot, the mobile device 622 can share its cellular Internet-connection 710 with the ex-vivo device 614 through a Wi-Fi connection 720. When providing a mobile hotspot, the mobile device 622 behaves as a router and provides a gateway to the cloud system 620. Also, as mentioned above, the mobile device 622 and the ex-vivo device 614 are capable of a Bluetooth® low energy (BLE) connection 730 for communicating control messages and/or data. A patient software app of the mobile device 622 can be used to set up the BLE connection 730 and/or the Wi-Fi connection 720 between the ex-vivo device 614 and the mobile hotspot of the patient mobile device 622. Various aspects of the patient app will be described later herein. FIG. 7 is exemplary, and variations are contemplated to be within the scope of the present disclosure.

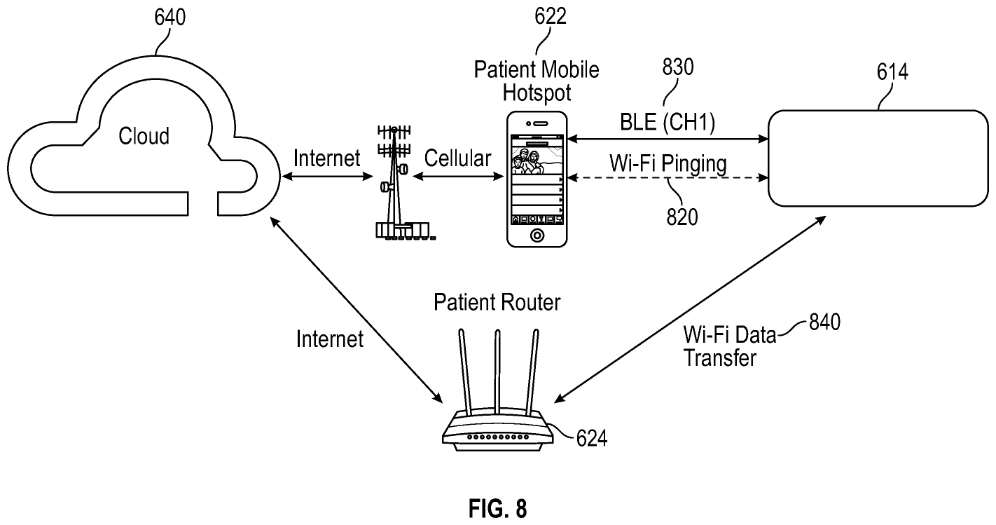

FIG. 8 shows a diagram of an exemplary communication path between an ex-vivo device 614 and a cloud system 640 via a communication device such as a router 624. When it is suitable for the ex-vivo device 614 to directly use a Wi-Fi network 840 (e.g., a home network), the patient can manually specify the Wi-Fi access credentials to the ex-vivo device 614 using a patient software app in the patient mobile device 622. Whenever the Wi-Fi network 840 is in range of the ex-vivo device 614, the ex-vivo device 614 can connect to the Wi-Fi network 840 and upload the procedure data via the communication device/router 624. In various embodiments, the ex-vivo device 614 can choose to simultaneously maintain a mobile hotspot Wi-Fi connection 820 and a router Wi-Fi connection 840.

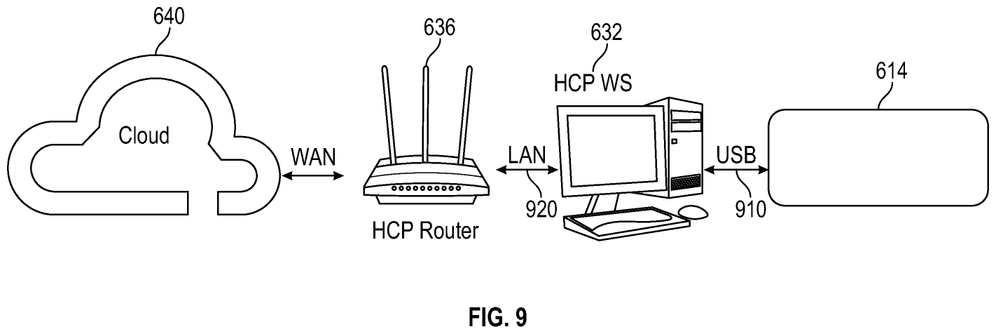

FIG. 9 shows a diagram of an exemplary communication path between an ex-vivo device 614 and a cloud system 640 via a healthcare provider workstation 632 of a medical facility. The illustrated communication path can be used whenever procedure data in the internal storage of the ex-vivo device 614 was not uploaded or not fully uploaded to the cloud system 640. The patient can provide the ex-vivo device 614, or a removable storage of the ex-vivo device 614, to the medical facility, and personnel at the facility can connect the ex-vivo device 614 or the removable storage to a workstation 632 via a USB connection 910. The procedure data is transferred from the ex-vivo device 614 to the workstation 632, and then the workstation 632 transfers the procedure data to the cloud system 640 using the facility's network infrastructure, such as a router 636 and local area network 920. A software application on the workstation 632 can coordinate the upload of procedure data to the cloud system 640. FIG. 9 is exemplary and does not limit the scope of the present disclosure. For example, in various embodiments, the healthcare provider workstation 632 can be a laptop computer or another device. Such variations are contemplated to be within the scope of the present disclosure.

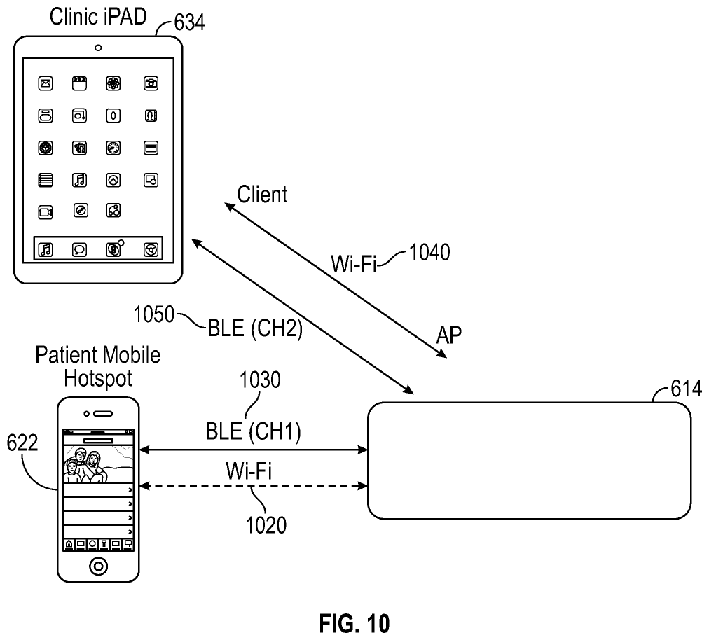

FIG. 10 shows a diagram of an exemplary direct connection between an ex-vivo device 614 and a healthcare provider device 634. By default, the ex-vivo device 614 can periodically connect to a Wi-Fi connection 1020 or Bluetooth Low Energy connection 1030 for data uploading. Whenever the ex-vivo device 614 receives a predetermined request, which will be referred to herein as a “real-time access” request, the ex-vivo device 614 changes its Wi-Fi setup from station to AP and permits a healthcare provider device 634 to establish a Wi-Fi connection 1040 or a Bluetooth Low Energy connection 1050 to the ex-vivo device 614. In summary, “real-time access” enables a healthcare provider device 634 to receive an immediate snapshot of recent procedure data by locally/directly connecting to the ex-vivo device 614. This functionality may be available during a procedure when the patient is in a medical facility. FIG. 10 and the described embodiments are exemplary, and variations are contemplated to be within the scope of the present disclosure. In various embodiments, the healthcare provider device 634 may not be a tablet and can be another type of device, such as a smartphone, laptop, or desktop computer, for example. Such variations are contemplated to be within the scope of the present disclosure.

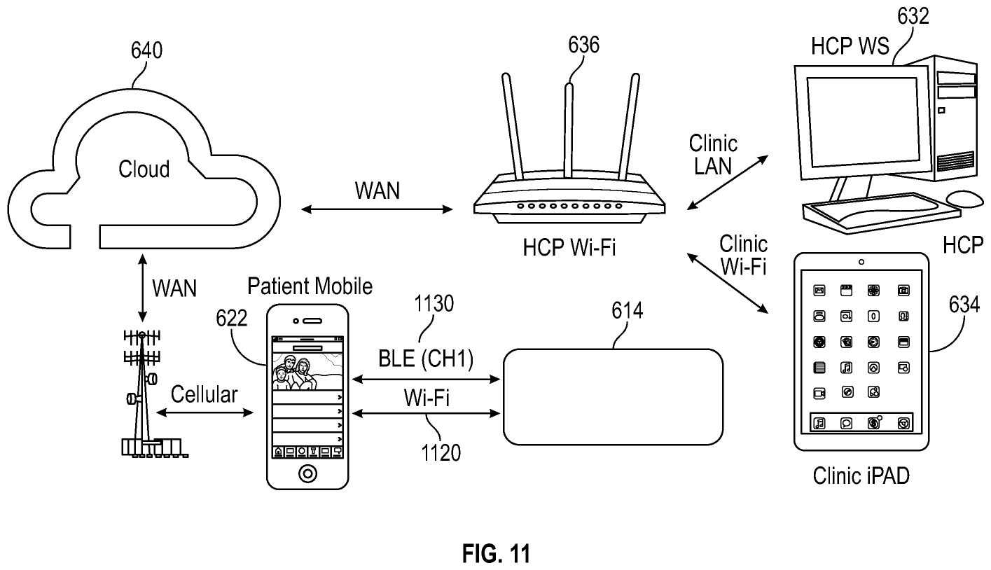

FIG. 11 shows a diagram of exemplary communication paths between an ex-vivo device 614 and healthcare provider devices 632, 634. The communication path between the ex-vivo device 614 and the cloud system 640 is the same as that described above in connection with FIG. 7 or can be the same as that illustrated in FIG. 8. The communication path between the healthcare provider devices 632, 634 and the cloud system 640 is a usual connection through a network infrastructure, such as a router 636. In accordance with aspects of the present disclosure, the healthcare provider (HCP) devices 632, 634 can include a software app that can initiate a command for the ex-vivo device 614, which will be referred to as a “near real-time access” command. The near real-time access command can be conveyed through the healthcare provider network infrastructure to the cloud system 640, which may send a corresponding command to the ex-vivo device 614 through the Wi-Fi connection 1120 or the BLE connection 1130 of the patient mobile device 622. In various embodiments, the command from the cloud system 640 can be an instruction for the ex-vivo device 614 to immediately upload the most recent procedure data which has not yet been uploaded to the cloud system 640. The cloud system 640 receives the procedure data upload and communicates the procedure data to the healthcare provider device 632, 634 so that a healthcare professional can review the latest procedure data in near real-time. Accordingly, this functionality, and its corresponding command, are referred to herein as “near real-time access.”

Accordingly, described above are various systems and devices and connections and communications between the systems and devices. In accordance with aspects of the present disclosure, the systems and devices disclosed above may operate to support a procedure performed by an in-vivo device, located in a person's GI tract, for taking measurements (e.g., pH measurements) to diagnose various esophageal or gastrointestinal diseases, such as gastroesophageal reflux disease (GERD), among others.

A disease evaluation may be aided by event information. In the example of GERD, an evaluation is based on using pH measurements to identify acid reflux events. Food or beverage consumption may directly affect measured pH levels, and exercise events may also affect the GI tract. Information about such and other events may help increase the accuracy of a GERD evaluation. As mentioned above in connection with FIG. 6 and FIG. 7, the patient mobile device 622 includes a software app. In accordance with aspects of the present disclosure, the software app of the mobile device 622 can operate to collect event information entered by a user via an input device (e.g., 535, FIG. 5) and can determine or acquire other event information without human intervention. Such event information is also encompassed within the term “procedure data” used herein.

As an example of event information determined without human intervention, and as described in connection with FIG. 4, movement sensor data may be used to automatically detect that an eating event is occurring or has occurred. In various embodiments, movement sensor data collected by a wearable device, such as by a smartwatch or bracelet, may be communicated from the wearable device to the mobile device 622 either directly or through one or more intermediate devices, such as through ex-vivo device 614. In various embodiments, the computational resources of the mobile device 622 may be sufficient for the software app to process the movement sensor data to determine whether an eating event is occurring or has occurred, as well as determine event information for such an event (e.g., start time and/or end time). Where the computational resources of the mobile device 622 may not be sufficient, the mobile device 622 may communicate the movement sensor data to the cloud system 640, where the cloud system 640 may process the movement sensor data. The cloud system 640 may determine whether an eating event is occurring or has occurred, as well as determine event information for such an event (e.g., start time and/or end time), and may communicate its determination back to the software app of the mobile device 622. The event information may be stored in the mobile device 622 and may be stored in the cloud system 640. In addition to such event information, the software app of the mobile device 622 may permit a user to enter other information, such as type of food or beverage consumed and/or an end time for the eating event, among other things. By having information about the times and contents of food or beverages consumed, such event information may aid in the evaluation of diseases such as GERD.

An eating event is merely illustrative, and other types of events are contemplated to be within the scope of the present disclosure, such as sleeping events and/or exercise events, among others. A sleeping event may cause greater reflux activity due to horizontal sleeping position and the corresponding position of the lower esophageal sphincter. Such events may be determined without human intervention, such as determined using movement sensor data, time of day, heart rate, and other data. Additional information about such events may be entered by a user using an input device. Such and other events, data, and information are encompassed within the term “procedure data” used herein and are contemplated with be within the scope of the present disclosure.

With continuing reference to FIG. 6, and in accordance with aspects of the present disclosure, a software app of the mobile device 622 may identify mistakes or errors in information entered by a user. For example, the software app may identify entered values that are impossible values and may be prompt a user to correct the mistake or error. In various embodiments, the software app may prompt a user for information when various data, such as pH data from the in-vivo device 612, indicates abnormal readings. The prompt may ask a user to indicate whether an event is occurring to cause the abnormal readings and, if so, enter event information for the event. In this manner, the software app may engage in a “dialogue” with the user to obtain correct information and/or further information. The description above is illustrative, and variations are contemplated to be within the scope of the present disclosure.

In accordance with aspects of the present disclosure, the evaluation of an esophageal or gastrointestinal disease may apply a trained machine learning model, such as deep neural network or a model which includes a deep learning neural network. A deep learning neural network is a machine learning model that does not require feature engineering. Rather, a deep learning neural networks can use a large amount of input data to learn correlations, such as learning correlations between input data and the presence or absence of an esophageal or gastrointestinal disease such as GERD.

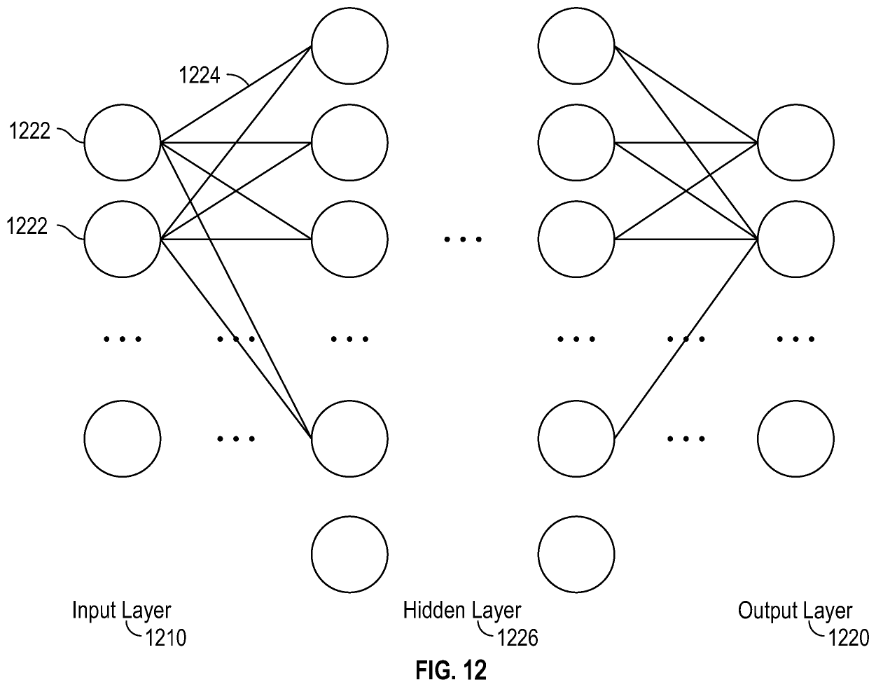

Referring to FIG. 12, a deep learning neural network includes an input layer 1210, a plurality of hidden layers 1226, and an output layer 1220. The input layer 1210, the plurality of hidden layers 1226, and the output layer 1220 are all comprised of neurons 1222 (e.g., nodes). The neurons 1222 between the various layers are interconnected via weights 1224. Each neuron 1222 in the deep learning neural network computes an output value by applying a specific function to the input values coming from the previous layer. The function that is applied to the input values is based on the vector of weights 1224 and/or a bias. Learning in the deep learning neural network progresses by making iterative adjustments to these biases and/or weights. Referring also to FIG. 6, the deep learning neural network may be trained and implemented by the cloud system 640.

In accordance with aspects of the present disclosure, a deep learning neural network may be trained to classify input data as indicative of GERD or as not indicative of GERD. In various embodiments, input data to the deep learning neural network may include all or a portion of pH measurements measured by an in-vivo device and/or may include event information for events such as eating events, sleep events, and/or exercise events, among others. In various embodiments, the input data may include temporal information, such as timing of pH measurements and/or timing of events, or may not include temporal information. Because feature engineering is not required for a deep learning neural network, the amount of input data used for the deep learning neural network may be permitted to be overinclusive, and the deep learning neural network may perform adequately without temporal information in the input data. Use of a deep learning neural network to indicate presence or absence of GERD, or of another esophageal or gastrointestinal disease, may save a healthcare provider time from not having to perform a manual analysis of pH data obtained by the in-vivo device. In various embodiments, the deep learning neural network may be trained using a cloud system, such as the cloud system 640 of FIG. 6. The result of applying the deep learning neural network may be provided to a device of a healthcare provider, such as the healthcare provider devices 632, 634 of FIG. 6.

In accordance with aspects of the present disclosure, use of a deep learning neural network may save the patient time by providing a diagnosis sooner. As mentioned above, existing esophageal devices may collect data for about 96 hours, and a diagnosis is provided after that data collection time period. In contrast, a deep learning neural network according to the present disclosure may process data during the course of the procedure, using any of the systems described in connection with FIGS. 6-11, and in various situations, may provide a diagnosis using data collected over twenty-four hours or less.

During the course of the procedure, and while the in-vivo device 612 is collecting data, the data may be relayed to the cloud system 640 through the ex-vivo-device 614 and one or more other devices, as shown in FIG. 6. If any event information is available from the patient mobile device 622, the event information may also be communicated to the cloud system 640. The cloud system 640 can implement multiple deep learning neural networks. For example, the cloud system 640 can implement a first deep learning neural network trained using data collected over twelve hours and implement a second deep learning neural network trained using data collected over sixteen hours, and so on and so forth. When data has been collected over twelve hours by the in-vivo device, that data may be input to the first deep learning neural network. If the first deep learning network is able to provide a classification that meets confidence criteria (e.g., using classification score thresholds), the procedure can be ended at that time. But if the first deep learning network is unable to provide a classification that meets confidence criteria, further data is collected by the in-vivo device and is communicated to the cloud system 640 until the second deep learning neural network can be applied to sixteen hours of collected data. If the second deep learning network is able to provide a classification that meets confidence criteria, the procedure can be ended at that time. But if the second deep learning network is unable to provide a classification that meets confidence criteria, further data is collected by the in-vivo device and is communicated to the cloud system 640 until the next deep learning neural network can be applied, and so on and so forth. In this manner, the present disclosure evaluates data during the course of a procedure and can provide a diagnosis in a shorter length of time. In various situations, it may not be appropriate to provide a diagnosis in less than twenty-four hours, such as when a diagnosis does not meet confidence criteria (e.g., based on threshold values). Deep learning neural networks or other machine learning models may be trained to use data collected over a longer time duration, such as forty-eight hours or another time duration. Such machine learning models may be used to provide a diagnosis using data collected by the in-vivo device over longer time periods.

Once the cloud system 640 provides a diagnosis and determines that the procedure can end, the cloud system 640 can communicate the decision to the patient mobile device 622. The decision that the procedure can end may cause the patient mobile device 622 to display a message that the ex-vivo device 614 can be removed because the procedure has ended. In various embodiments, the cloud system 640 and/or the patient mobile device 622 may communicate an instruction to the ex-vivo device 614 to cause the ex-vivo device 614 to stop operating and/or communicate an instruction to the in-vivo device 612 to cause the in-vivo device 612 to stop operation. Such embodiments are illustrative, and other embodiments and variations are contemplated to be within the scope of the present disclosure.

Accordingly, the description above describes collecting event information, applying deep learning neural networks to diagnose esophageal or gastrointestinal diseases, and processing data during a procedure to provide a diagnosis using data collected over twenty-four hours or less. The description is illustrative, and variations are contemplated to be within the scope of the present disclosure. For example, referring to FIG. 10, a “real-time access” command may be used by a healthcare provide device 634 to immediately access data from the ex-vivo device 614, if the patient is located at the healthcare provider facility. Such a command may allow a healthcare provider to, for example, determine whether the in-vivo device is functioning properly, among other things. If the patient is not located at the healthcare provider facility, then as described in connection with FIG. 11, a “near real-time access” command may be used by a healthcare provider device 632, 634 to access data from the ex-vivo device 614. Such a command may allow a healthcare provider to, for example, remotely determine whether the in-vivo device is functioning properly, among other things. Such and other embodiments and variations are contemplated to be within the scope of the present disclosure.

In accordance with aspects of the present disclosure, and referring to FIG. 6, the cloud system 640 can store and analyze procedure data for multiple patients. The cloud system 640 can analyze such data to provide personalized recommendations for a patient. The personalized recommendation can be based on analysis of procedure data specific to the patient. In various embodiments, the personalized recommendation can be based on procedure data of other patients who share a common characteristic with the patient. The personalized recommendation may include, for example, a proposed personalized diet that mitigates GERD by consuming or avoiding certain food or beverage items and/or by consuming or avoiding certain food or beverage items according to a proposed schedule. Such and other embodiments are contemplated to be within the scope of the present disclosure.

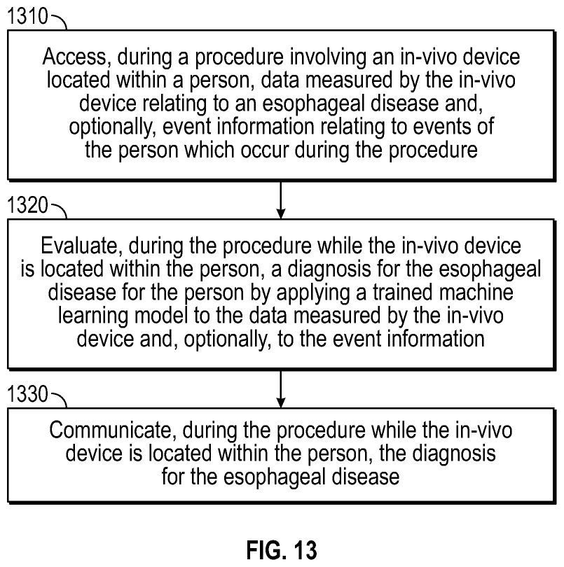

FIG. 13 is a flow diagram of an exemplary operation for diagnosing an esophageal disease, such as GERD. The illustrated operation may be performed by, for example, a cloud system, such as the cloud system 640 of FIG. 6. Depending on available computing resources, the operation may be performed by another system or device.

The operation of FIG. 13 relates to a procedure involving an in-vivo device located within a person and is performed during the procedure while the in-vivo device is located within the person. At block 1310, the operation involves accessing data measured by the in-vivo device relating to an esophageal disease and, optionally, accessing event information relating to events of the person which occur during the procedure. In various embodiments, the esophageal disease may be GERD, and the data measured by the in-vivo device may be pH measurements. In various embodiments, the optional event information may include information on an eating event or a sleep event for the person. In various embodiments, some or all of the event information may not be entered by the person and may, instead, be generated by a mobile device of the person or by a wearable device worn by the person.

At block 1320, the operation involves evaluating a diagnosis for the esophageal disease for the person by applying a trained machine learning model to the data measured by the in-vivo device and, optionally, to the event information. In various embodiments, the trained machine learning model may be a trained deep learning neural network. As described above, the deep learning neural network may be trained to classify input data as indicating presence of the esophageal disease or absence of the esophageal disease. In various embodiments, the trained deep learning neural network may be configured to be applied to data collected over a predetermined time duration that is less than twenty-four hours. In various embodiment, a diagnosis may be provided only if it meets confidence criteria.

At block 1330, the operation involves communicating the diagnosis for the esophageal disease. In various embodiments, the diagnosis may be communicated to a healthcare provider for the healthcare provider to, then, explain to the patient. In various embodiments, the diagnosis may be available within twenty-four hours of the procedure being initiated, while the in-vivo device is still within the patient. Once a diagnosis is available, the patient may be notified that the procedure has ended, and any wearable equipment associated with the procedure may be removed. FIG. 13 is illustrative, and variations are contemplated to be within the scope of the present disclosure.

Those skilled in the art to which this disclosure pertains will readily appreciate that numerous changes, variations, and modifications can be made without departing from the scope of the disclosure, mutatis mutandis.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

The systems, devices, and/or servers described herein may utilize one or more processors to receive various information and transform the received information to generate an output. The processors may include any type of computing device, computational circuit, or any type of controller or processing circuit capable of executing a series of instructions that are stored in a memory. The processor may include multiple processors and/or multicore central processing units (CPUs) and may include any type of device, such as a microprocessor, graphics processing unit (GPU), digital signal processor, microcontroller, programmable logic device (PLD), field programmable gate array (FPGA), or the like. The processor may also include a memory to store data and/or instructions that, when executed by the one or more processors, cause the one or more processors to perform one or more methods and/or algorithms.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, Python, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A system for diagnosing an esophageal disease, the system comprising:
at least one processor; and
at least one memory storing instructions which, when executed by the at least one processor, cause the system to:
access, during a procedure involving an in-vivo device located within a person, data measured by the in-vivo device relating to an esophageal disease;
evaluate, during the procedure while the in-vivo device is located within the person, a diagnosis for the esophageal disease for the person by applying a trained machine learning model to the data measured by the in-vivo device; and
communicate, during the procedure while the in-vivo device is located within the person, the diagnosis for the esophageal disease.

2. The system of claim 1, wherein the instructions, when executed by the at least one processor, further cause the system to:
access, during the procedure while the in-vivo device is located within a person, event information relating to events of the person which occur during the procedure,
wherein evaluating the diagnosis for the esophageal disease for the person comprises applying the trained machine learning model to the data measured by the in-vivo device and to the event information.

3. The system of claim 2, wherein accessing the event information relating to events of the person which occur during the procedure comprises receiving the event information from a mobile device of the person,
wherein at least a portion of the event information is not entered by the person and is generated by at least one of: the mobile device of the person or a wearable device separate from the mobile device.

4. The system of claim 1, wherein the trained machine learning model comprises a trained deep learning neural network configured to be applied to data collected over a predetermined time duration that is less than twenty-four hours,
wherein the data measured by the in-vivo device comprises data measured by the in-vivo device over at least the predetermined time duration, such that the trained deep learning neural network is applied to the data measured by the in-vivo device over at least the predetermined time duration.

5. The system of claim 1, wherein the trained machine learning model is one model among a plurality of trained machine learning models, wherein models of the plurality of trained machine learning models are configured to be applied to data collected by the in-vivo device over different predetermined time durations.

6. The system of claim 5, wherein in evaluating the diagnosis for the esophageal disease for the person, the instructions, when executed by the at least one processor, cause the system to:
evaluate, at a first time during the procedure while the in-vivo device is located within the person, a first diagnosis for the esophageal disease for the person using a first model of the plurality of trained machine learning models;
determine that the first diagnosis does not meet confidence criteria;
evaluate, at a second time during the procedure while the in-vivo device is located within the person, a second diagnosis for the esophageal disease for the person using the trained machine learning model, wherein the second time is after the first time;
determine that the second diagnosis meets confidence criteria; and
provide the second diagnosis as the diagnosis for the esophageal disease for the person.

7. A computer-implemented method for diagnosing an esophageal disease, the method comprising:
accessing, during a procedure involving an in-vivo device located within a person, data measured by the in-vivo device relating to an esophageal disease;
evaluating, during the procedure while the in-vivo device is located within the person, a diagnosis for the esophageal disease for the person by applying a trained machine learning model to the data measured by the in-vivo device; and
communicating, during the procedure while the in-vivo device is located within the person, the diagnosis for the esophageal disease.

8. The computer-implemented method of claim 7, further comprising:
accessing, during the procedure while the in-vivo device is located within a person, event information relating to events of the person which occur during the procedure,
wherein evaluating the diagnosis for the esophageal disease for the person comprises applying the trained machine learning model to the data measured by the in-vivo device and to the event information.

9. The computer-implemented method of claim 8, wherein accessing the event information relating to events of the person which occur during the procedure comprises receiving the event information from a mobile device of the person,
wherein at least a portion of the event information is not entered by the person and is generated by at least one of: the mobile device of the person or a wearable device separate from the mobile device.

10. The computer-implemented method of claim 7, wherein the trained machine learning model comprises a trained deep learning neural network configured to be applied to data collected over a predetermined time duration that is less than twenty-four hours,
wherein the data measured by the in-vivo device comprises data measured by the in-vivo device over at least the predetermined time duration, such that the trained deep learning neural network is applied to the data measured by the in-vivo device over at least the predetermined time duration.

11. The computer-implemented method of claim 7, wherein the trained machine learning model is one model among a plurality of trained machine learning models, wherein models of the plurality of trained machine learning models are configured to be applied to data collected by the in-vivo device over different predetermined time durations.

12. The computer-implemented method of claim 11, wherein evaluating the diagnosis for the esophageal disease for the person comprises:
evaluating, at a first time during the procedure while the in-vivo device is located within the person, a first diagnosis for the esophageal disease for the person using a first model of the plurality of trained machine learning models;
determining that the first diagnosis does not meet confidence criteria;
evaluating, at a second time during the procedure while the in-vivo device is located within the person, a second diagnosis for the esophageal disease for the person using the trained machine learning model, wherein the second time is after the first time;
determining that the second diagnosis meets confidence criteria; and
providing the second diagnosis as the diagnosis for the esophageal disease for the person.

13. A computer-readable medium comprising instructions which, when executed by at least one processor of a system, cause the system to:
access, during a procedure involving an in-vivo device located within a person, data measured by the in-vivo device relating to an esophageal disease;
evaluate, during the procedure while the in-vivo device is located within the person, a diagnosis for the esophageal disease for the person by applying a trained machine learning model to the data measured by the in-vivo device; and
communicate, during the procedure while the in-vivo device is located within the person, the diagnosis for the esophageal disease.

14. The computer-readable medium of claim 13, wherein the instructions, when executed by the at least one processor, further cause the system to:
access, during the procedure while the in-vivo device is located within a person, event information relating to events of the person which occur during the procedure,
wherein evaluating the diagnosis for the esophageal disease for the person comprises applying the trained machine learning model to the data measured by the in-vivo device and to the event information.

15. The computer-readable medium of claim 13, wherein the trained machine learning model comprises a trained deep learning neural network configured to be applied to data collected over a predetermined time duration that is less than twenty-four hours,
wherein the data measured by the in-vivo device comprises data measured by the in-vivo device over at least the predetermined time duration, such that the trained deep learning neural network is applied to the data measured by the in-vivo device over at least the predetermined time duration.
